(19) United States
(12) Patent Application Publication
(10) Pub. No.: US 2011/0067698 A1
(43) Pub. Date: Mar. 24, 2011

(54) HANDHELD DEVICE FOR DELIVERING CONTINUOUS POSITIVE AIRWAY PRESSURE

(75) Inventors: Bing ZHENG, Etobicoke (CA); Ammar AL-DOJAILY, Milton (CA); Kevin D. J. BOWDEN, Orangeville (CA)

(73) Assignee: O-TWO MEDICAL TECHNOLOGIES INC., Mississauga (CA)

(21) Appl. No.: 12/887,919

(22) Filed: Sep. 22, 2010

Related U.S. Application Data
(60) Provisional application No. 61/244,721, filed on Sep. 22, 2009.

(51) Int. Cl. A61M 16/20; A61M 16/00

(52) U.S. Cl. 128/204.25; 128/205.24

(57) ABSTRACT

A hand held CPAP device for providing a continuous positive airway pressure where pressure is generated by a flow of gas from a demand valve by the interaction of an adjustable compression spring compressing the demand valve actuating diaphragm.

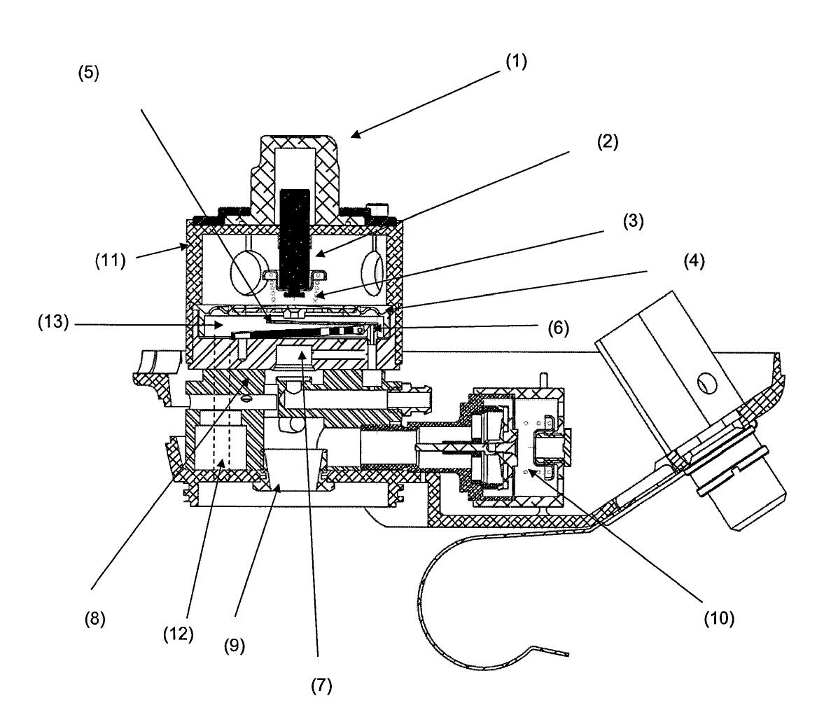

### TECHNICAL FIELD

The invention relates to a hand held CPAP device for providing a continuous positive airway pressure where pressure is generated by a flow of gas from a demand valve by the interaction of an adjustable compression spring compressing the demand valve actuating diaphragm

### BACKGROUND TO THE ART

Continuous Positive Airway Pressure (CPAP) is used for a number of applications in assisting patients with respiratory difficulties (such as Pulmonary Edema) or for persons suffering from sleep apnea. In the pre-hospital emergency market CPAP is becoming a primary treatment option for many respiratory impaired patients. Current CPAP devices are large, cumbersome and heavy. The invention that is the subject of this application is lightweight and handheld.

### DISCLOSURE OF THE INVENTION

The invention provides a device for providing a continuous positive airway pressure where the pressure is generated by a flow of gas from a demand valve.

### DESCRIPTION OF THE DRAWINGS

The attached drawing is a sectional view along a plane through a central axis of the device

### DESCRIPTION OF THE CURRENT INVENTION

The new device utilizes a simple rotating control (1) to interface with a threaded member (2) which rotates within a housing (11) to compress a spring (3) against a diaphragm (4) which in turn depresses a tilt lever (5) within the body of the device to open a jet orifice (6). The flow of gas from the jet orifice lowers the pressure in the control chamber (7) producing a flow of gas from the demand valve (8). This gas flow passes through conduit (9) to a circuit means to deliver the flow to a facemask attached to a patient (not shown). The constant flow of gas from the demand valve creates a positive pressure within the facemask.

On inspiration by the patient a conduit (12) within the body of the demand valve allows negative pressure to build up in tilt lever chamber (13) below the diaphragm which draws the diaphragm (4) downwards against the tilt lever (5) increasing the flow of gas from the jet orifice (6), dropping the pressure further in control chamber (7) and providing an increased flow of gas from demand valve (8) equivalent to that demanded by the patient. Because jet orifice (6) is already opened by the actuation of the CPAP control, the negative pressure required to be generated is lower than that normally required to “crack” the tilt lever seal on the jet orifice thereby not allowing the pressure to enter a negative phase providing the CPAP pressure.

To prevent a dangerous build up of pressure in the event of a device failure a pressure relief system (10) is incorporated into the design.

1) A device for providing a continuous positive airway pressure whereby said pressure is generated by a flow of gas from a demand valve.

2) A device according to claim 1, for providing a continuous positive airway pressure whereby said device is designed to be hand held.

3) A device according to claim 1, for providing a continuous positive airway pressure whereby said flow of gas from the demand valve is provided by the interaction of a spring compressing the demand valve actuating diaphragm to actuate the tilt lever to open the jet orifice, dropping the pressure in the control chamber and generating a positive flow of gas from the demand valve.

4) A device according to claim 1, for providing a continuous positive airway pressure whereby said spring compression is generated by the rotary motion of a control knob interacting with a threaded rod which rotates within a housing in turn interfaces with the spring. Increasing rotation of the control knob increases the pressure on the spring which in turn increases the pressure on the tilt lever, allowing more flow from the jet orifice and increasing the positive airway pressure generated.

5) A device according to claim 1, for providing a continuous positive airway pressure incorporating a pressure relieving mechanism to prevent over pressurization of the patent's airway in the event of a device failure.
